(19) United States
(12) Patent Application Publication
(10) Pub. No.: US 2023/0298694 A1
(43) Pub. Date: Sep. 21, 2023

(54) NON-ANIMAL HUMAN RELEVANT WORKSTATION SYSTEM AND METHOD FOR TESTING NEUROVIRULENCE AND NEUROTOXICITY IN VACCINES

(71) Applicants: Subhadra Dravida, Hyderabad (IN); Samhita B L N, Frisco, TX (US)

(72) Inventors: Subhadra Dravida, Hyderabad (IN); Samhita B L N, Frisco, TX (US)

(73) Assignee: TRANSCELL ONCOLOGICS PRIVATE LIMITED, Hyderabad (IN)

(21) Appl. No.: 17/722,528

(22) Filed: Apr. 18, 2022

(30) Foreign Application Priority Data
Feb. 15, 2022 (IN) 202241008032

(51) Int. Cl. G16B 20/50; G16B 40/00; G16C 20/30; C12Q 1/6827; C12Q 1/70; C12N 5/074

(52) U.S. Cl. CPC G16B 20/50; C12N 5/0696; C12Q 1/6827; C12Q 1/70; G16B 40/00; G16C 20/30

(57) ABSTRACT

A system and method for test predicting human neurovirulence and neurotoxicity risks is disclosed. The system comprises a real-time platform or TRANS-MSC (Configured Human induced Pluripotent Stem Cells) unit and a trained digital platform. The TRANS-MSC incubates the vaccine/biologic, drug/API, cosmetic/ingredient, anti-venom aliquots collected from the produced batches in the manufacturing system. The digital platform is embedded with artificial intelligence (AI) and machine learning (ML) modules, augmented with a robotic process automation framework. The AI modules predict human neurovirulence, human neurotoxicity patterns along with any adventitious microbial contaminants in the process. The AI and ML modules are trained with a plurality of TRANS-MSC acquired phenotype micrographs and a plurality of neurotoxic genes involved in viral, bacterial, fungal infections. Further, the test is customized to a genetically distinct population, user’s library of research-grade, ingredients, intermittents, final products, etc. that are at the risk of causing neurovirulence or neurotoxicity in the clinics.

### CROSS-REFERENCE TO RELATED APPLICATION

The present application claims priority to the Indian patent application No. IN 202241008032 filed Feb. 15, 2022, entitled “A Non-Animal Cruelty Free Human Relevant Workstation System and Method for Testing Neurovirulence and Neurotoxicity in Vaccines”, with the entire content of application incorporated herein by reference.

### FIELD OF THE INVENTION

The present invention generally relates to the field of any structural or functional mimic of neurotropic virus or viral component developed as vaccines. More specifically, the present invention relates to a system and method for cruelty-free testing of safety risks such as neurovirulence, neurotoxicity, and sterility in vaccines production process.

### BACKGROUND

A vaccine is a biological preparation that provides immunity to a particular infectious disease. Vaccines greatly reduce the risk of infection by working with the body’s natural defenses to safely develop immunity to disease. The vaccine contains an agent that resembles a disease-causing microorganism and is often made from weakened or killed forms of the microbe, its toxins, or one of its surface proteins. The agent stimulates the body’s immune system to recognize the agent as a threat, destroy it, and to further recognize and destroy any of the microorganisms associated with that agent that it may encounter in the future.

A report states that the vaccines have been the single most fruitful investment in public health after sanitation and clean water and have contributed to drastically reducing the incidence and mortality ensuing from pathogenic and communicable diseases. It is no wonder that some of the world’s largest philanthropies have invested heavily in these instruments, alongside state public health agencies. New pathogens will emerge in every era, and vaccines are likely to be the best bet against them. Nowhere has this been so starkly demonstrated as in the COVID-19 pandemic.

Vaccines are of various types, and their variety has proliferated in tandem with advances in the chemical, physical, biological sciences, and computational sciences. These comprise, in addition to the conventional types such as attenuated and live inactivated viral vaccines, viral-vectored vaccines, and subunit vaccines, newer forms like the viral-like particle vaccines, nanoparticle vaccines, recombinant proteins, polysaccharide-based immunogens, hybrid molecules (part engineered and part native), DNA/RNA vaccines, and the rationally-designed vaccines. Vaccine-induced prophylaxis is now available for non-communicable diseases (NCDs) such as diabetes, cancer, rheumatoid arthritis, and cardiovascular disease.

The newer vaccines are designed to be immunogenic and to simultaneously reduce the likelihood of adverse events. Their presence notwithstanding, inactivated and the live attenuated virus (LAV) vaccines continue to be part of the prophylactic arsenal of state public health services, as they are known to be effective, and industrial systems are already set up for their manufacture. The LAV comprises “live” viruses that can reproduce normally but have lost the ability, through mutations, to cause disease. Because they can multiply and amplify their titre in the host circulation over a period, the immune challenge is large enough, and long enough, for a single dose to suffice. Their ability to multiply in the host confers on them a peculiar property, and this is described below.

### Neurotropic Viruses and Neurovirulence

Viruses are fastidious beings, in that they target and affect specific tissues in specific organisms. This affinity to a specific tissue, in a specific host, is termed tropism. Neurotropic viruses therefore are those which selectively infect nervous tissue; they have evolved genetically to perform the various preliminary steps to successful neuroinvasion, and subsequent multiplication within neural or neuronal cells, to cause neurovirulence. Neurotropic viruses may enter the central nervous system through the blood-brain barrier, or “centripetally” through the peripheral nervous system. They may enter neuronal cells and establish latency, or cause apoptotic neural damage through the lytic pathway as shown in FIG. 1. Neurotropic viruses must counter the host’s innate immune response to infection, inhibit autophagy of cells they infect, and reverse the host-directed shutdown of the protein synthetic machinery, and there are variations on this theme, depending on the virus. Because neurovirulence targets the central nervous system (CNS) it often manifests clinically as encephalitis.

The list of neurotropic viruses is veritable who’s who of the most pernicious pathogens known to man. Cytomegalovirus, influenza viruses (including the human coronaviruses), and the viruses causing polio, Yellow Fever, Japanese Encephalitis, mumps, measles, rabies, herpes, and HIV. A large proportion of emerging viruses are neurotropic and can cause serious neurological disease, SARS-COV-2 causing Covid-19 being the most recent addition to this morbidly important group.

FIG. 1 shows the tendency or capacity of the vaccine to cause or trigger disease of the nervous system 100. The neurons 102 in the CNS are affected by SARS-COV and SARS-COV-2 virus 104 causing viral invasion in the CNS. The SARS-COV and SARS-COV-2 virus 104 affect the cell membrane 106 causing neural infection, where the cell membrane comprises an angiotensin-converting enzyme 2 (ACE2) 108 and a cell surface protein transmembrane serine protease 2 (TMPRSS2) 110. The virus entered the nervous systems cause neural damages 112 i.e., Immune-mediated CNS damage in the CNS.

### Theory Based on Genetics

A live attenuated vaccine virus that occasionally reverts to its original neurovirulent form is a public health hazard and is a serious impediment to vaccine use. Reversion is frequent enough in LAVs, and damaging enough, to warrant the development of safely attenuated LAV vaccines, and of reliable ways of assaying for this property. Revertant forms of the viral vaccine may cause neurological adverse events (AEs) ranging from fever to paralysis to death. This concern has historical basis.

The Guillain Barre syndrome, caused by Johnson and Johnson’s single-shot vaccine is the most recent instance, of several, vaccine-derived neurovirulence. The neurotropic virus vaccines introduced into the market since 2001 number more than 60, and underscore the importance of this safety check. For this reason, international and regional laws require that vaccine lots be assiduously checked for neurovirulence before releasing for sale and that pharmacovigilance be enforced. A reliable testing mechanism for reacquisition of neurovirulence in the vaccine batch production will go far in mitigating this problem.

Mutations responsible for live-virus attenuation have not been fully characterized, except for the oral polio vaccine (OPV). Viruses have much higher rates of mutations than do bacteria or higher life forms, and RNA viruses more than DNA viruses. Attenuation requires different numbers of passages for different viruses, and this is peculiar to the virus; some require a few tens, others several hundred. Further, different numbers of attenuating mutations are present in different live attenuated viruses, and those with a larger number are less likely to revert. The quality that confers attenuation can also cause spontaneous reversion to the virulent wildtype form. Evolution cuts both ways. A reversion may be through back-mutations, compensatory mutations in different regions of the genome, and recombination with other viruses. About 1 in 750,000 children who are vaccinated with the OPV are afflicted with vaccine-derived polio. Studies with the oral polio vaccine have also shown that reversion rates are appreciable and that they depend on the immunization schedule and the route of administration of the vaccine. The propensity of the virus to revert to a virulent form is such that the live-attenuated form of the poliovirus vaccine has been completely replaced by the inactivated form.

A systematic examination of the genetic basis for neurovirulence in highly neurovirulent and attenuated strains was done in the mumps virus using recombinant DNA; the work indicated that several rJL genes and gene combinations were responsible for neuroattenuation. The work also suggested that there are mechanistic differences in the way a strain acquires or loses, neurovirulence. Systematic analyses of the vaccinia virus’s neurovirulence have also been done, and its neurovirulence genes appear to be expressed in synchrony with those regulating the virus’s infective and invasive processes. Genetic control of neurovirulence of the Herpes Simplex Virus has been traced to the γ34.5 gene or neurovirulence factor, although other genes are believed to be involved in an auxiliary capacity. In the Influenza virus, neurovirulence is determined by several mutations in the coding regions of the haemagglutinin (HA), neuraminidase (NA), matrix (M), and non-structural (NS) genes. Genetic changes in the neurovirulent influenza strains are observed in the neurovirulent variants of other viruses, suggesting that there are a common set of strategies that viruses use to infect the CNS, replicate, and cause neurovirulence. Neurovirulence in the Osaka-2 strain of the Measles virus is caused by the F gene, and a specific mutation in the same gene is responsible for reversion to neurovirulence. The mumps virus (MuV) is neurotropic and highly neurovirulent, and one of the major causes of encephalitis in the Western Hemisphere but the culprit genes are not known.

Because live attenuated viral vaccines (LAVs) are still more efficacious than the subunit or recombinant vaccines, and because reversion reduces the safety of live virus vaccines and their utility, research has of late focused on the directed and permanent attenuation of virulence (including neurovirulence) in the virus. Several methods have been explored for engineering in fail-proof mechanisms of attenuation, as safeguards against stochastic reversion to wild-type forms. These directed attenuation schemes include deleterious gene mutations, altered replication fidelity, codon de-optimization, and micro-RNA or Zinc Finger Nuclease control. Care is taken to ensure that alterations done to eliminate the possibility of future reversion do not simultaneously abolish the virus’s immunogenic potential. Live attenuated influenza vaccines, for instance, have been created by the genetic alteration of the trypsin cleavage site to an elastase cleavage site. The resulting virus is genetically homologous to the wild type and does not pose any danger of reversion.

### Manufacture of the Live Attenuated Viral Vaccine

As the viruses are obligate parasites, they can only be cultured in cellular hosts; Vaccine viruses are “grown” in specific human or animal cell lines, in embryonated eggs, in animals (such as primates), or in animal- animal embryonic tissue. Chicken embryos serve as the cellular substrate for the culture of several LAVs, including the influenza virus. Chicken embryos are grown in fertilized eggs until they reach a certain size; they are harvested and treated with Trypsin to break up the tissue into individual cells (fibroblasts), which are then cultured in roller bottles, in media supplemented with fetal calf serum, such as the M199 Hank’s media, for the measles virus. Embryonal cells are infected with the viral working lots and cultured for a predetermined period of time. Cells are collected, separated from media, and lysed to release viral particles; the latter is in turn collected by centrifugation, purified, and reused as inoculum. The virus is put through several iterations (passages) of this process, from a few dozen to a few hundred, and this process is variously modified and refined, depending on the virus, to improve yield.

This method of viral culture was first employed by Louis Pasteur, and it is based on the principle that the adaptations of the virus to a new host (the substrate, in this case) weakens its ability to replicate in the native host. The virus, during the passages through the new host, accumulates mutations that gradually weaken and eliminate its quality of virulence, including neurovirulence; By the same logic, the virus tends to re-acquire the virulent property when it is introduced into its natural host as a vaccine. RNA genomes evolve faster than DNA genomes because they accumulate a greater number of mutations per replicative cycle (or passage). In this traditional way of manufacture, the attenuated phenotype indicates corresponding changes in the genotype. The exact causal changes in genetic code have not been fully characterized, except, as mentioned above, for the oral polio vaccine.

After the desired attenuated viral isolate is prepared, it is expanded to develop the Master Lots. Master seed lots of neuroattenuated viruses are examined for neurovirulence by the MNVT and comprise the primary reagent in the vaccine’s manufacture. Major regulatory agencies today require at least five Master seed lots to be tested for Neurovirulence by the MNVT before vaccine production may commence. The Master seed virus stock is used to generate a larger Working virus stock that is then cultured in the appropriate cellular substrate, as described above, for mass production of vaccine. When the working stock is exhausted, and this takes many years, it is regenerated using an aliquot of the master viral seed stock.

The following live attenuated vaccines are currently in use worldwide: vaxchora for cholera, Flumist for influenza, M-M-R II for Measles, Mumps, Rubella, Pro-Quad for Measles, Mumps, Rubella, Attenuvax for Measles, M-M-Vax for Measles and Mumps, Mumpsvax for Mumps, Vivotif for Typhoid, Varivax for Varicella (chickenpox), Zostavax for Zoster (Varicella), ACAM2000 for Vaccinia (smallpox), YF-VAX for Yellow Fever, JYNNEOS for Smallpox and Monkeypox, Sabin vaccine for Polio, Rotarix for Rotavirus, BCG for Tuberculosis, etc.

From proof-of-principle to an IND (Investigational New Drug), to clinical trials, to final licensure, and pharmacovigilance post-licensure, vaccines tread the same regulatory path as drugs. It is important that safety assessments cover the duration of the vaccine’s life-cycle, through the progression from basic research studies to product and product use, as a reversion to neurovirulence may happen at any time. In India, regulatory oversight for drugs, cosmetics, biologics, and medical devices is the responsibility of the Central Drug Standards Control Organization (abbrev.CDSCO); the agency operates under the aegis of the Ministry of Health and Family Welfare (MoHFW). The American CDC’s Center for Biologics Evaluation and Research (CBER), the European Medicines Agency (EMA), and the Japanese PMDA (Pharmaceuticals and Medical Devices Agency) are other major regulatory agencies. The International Conference on Harmonization (or ICH) endeavors to make uniform national and international regulatory policy, and to thereby facilitate drug development, manufacture, sale, and surveillance. Post-licensure, pharmacovigilance mechanisms such as the Adverse Events Following Immunization program (AEFI, in India) and the Vaccine Adverse Event Reporting System (VAERS, in the United States) surveil vaccine use for safety concerns post-licensure.

### The Monkey Neurovirulence Test Methodology

If viruses intended for vaccine manufacture are naturally neurotropic, or bear components that are neurotropic, or have been passaged through neuronal cells, regulations require that they be assessed for not just general virulence, but also neurovirulence. Neuroattenuation must be assessed and demonstrated, and this generally takes the form of the Monkey Neurovirulence Test (MNVT) as shown in FIG. 2.

To reiterate, the vaccine Master Lots are required by law to be tested by the MNVT; the USFDA requires that 5 of these Master Lots be tested for neuroattenuation, or neurovirulence. Subsequent lot testing is generally not followed once the master lots are checked. The frequency of testing post the Master Lot checks depends on the regulatory agency: the World Health Organisation (WHO), the Japanese PMDA, the American Food and Drug Administration (USFDA), or the EMA.

The MNVT takes the form of a regular experiment. Each test requires approximately 30 monkeys; three groups of monkeys are used, one as a negative control that does not receive any virus, one as a positive control that receives a virulent form, and one test group that receives the candidate vaccine. Candidate virus is inoculated into the brain or spinal cord of monkeys of the Macaca or Cercopithecus genera and animals observed for symptoms of neural damage over a 17 to 22-day period. Monkeys were traditionally monitored for clinical signs of encephalitis, and this practice continues. In addition, the monkeys are also euthanized and examined by histopathology for viral lesions in the brain and spinal cord tissue. Three regions of the brain are of interest: the target regions, which are inflamed upon infection by both neurovirulent and non-neurovirulent viruses, the eponymous discriminator regions, which are preferentially infected by the neurovirulent viruses, and control regions that are not affected. The degree of neurovirulence is inferred from glial cell activation and infiltration of the CNS by primary immune cells, and these cellular events are semi quantitatively scored.

The MNVT as it is called, has been the neurovirulence test of choice for poliomyelitis, measles, mumps, rubella, varicella, influenza, yellow-fever viruses, and more recently for the COVID vaccines, the rationale for the model being the phylogenetic proximity of human and nonhuman primates. The rationale is questionable; there is a certain dissonance between the MNVT’s persistence in vaccine safety testing and the fundamental flaw in it, viz that genetic relatedness does not always translate into the relevance and predictive value (although this is a general caveat with any model system). A lack of a resemblance between the simian cell surface structures, which the virus uses to gain entry, can preclude the MNVT’s relevance to the virus and render it useless. Instances of the MNVT’s inability to detect neurovirulence exist. For mumps vaccines, the MNVT only showed non-significant trends towards differences between wild-type and attenuated mumps viruses and failed to detect residual neurovirulence in the Urabe Am9 strain of mumps vaccine. This strain was developed in 1967 based on a Japanese isolate of mumps virus, passaged through chicken and quail cells. The vaccine was widely distributed in Canada, Japan, and Europe until cases of vaccine-associated aseptic meningitis were detected in Canada. More cases were found in Japan and the UK, with an estimated 38-330 cases of aseptic meningitis per 100,000 vaccine recipients. Following these findings, the Urabe Am9 vaccine saw reduced use, and in Japan, mumps was removed as a routine vaccine altogether. Following this policy change, Japan has seen a surge in mumps cases, with up to 1.5 million infections annually.

The non-translational results of the MNVT led to the scientific community revisiting the value of the MNVT for vaccine safety testing, and in 2005, the International Alliance for Biological Standardization (IABS) released a report on neurovirulence tests for live attenuated vaccines. This workshop report concluded that the monkey neurovirulence test was useful for testing YFV and poliovirus vaccines but was questionably useful for most of the viruses for which it is currently used, including mumps, measles, rubella, influenza, varicella, and the Yellow Fever virus is one of the few exceptions. The MNVT checks for a single end-point, either clinical encephalitis or viral lesions in neuronal tissue, that may not reflect the various cellular mechanisms by which viruses can cause neurovirulence. The use of multiple human-specific molecular or cellular end-points would better indicate these various mechanisms and endow a neurovirulence test with greater predictive value. Furthermore, the use of live animals as test material is abhorrent to many individuals, on ethical and humane grounds, and public sentiment and state policy have discouraged the use of these in the drug/ biologic or cosmetic testing enterprise. Research on murine animal models has been less affected by such sentiment, although these still have the limitation of translatability.

### Alternatives to the MNVT

The MNVT was conceived at a time when comparable model systems did not exist, but the scientific and technological context has changed now, and alternatives should be given serious consideration. Efforts have been ongoing for years now to expand the repertoire of alternatives, but R&D alternatives to the MNVT have been slow coming. The primary reason is technical: it is difficult to develop a model that resembles the real “thing” (in this case, the human) to the degree that it is predictive of the actual response. Monkeys and (transgenic) mice are good models to the degree that their cellular and molecular anatomy resembles the humans’, i.e., if the cell-surface structures facilitating viral entry are the same in the two. The technical problems notwithstanding, non-human primates other than monkeys have been used, successfully, for some vaccines, including the Ebola virus, Zika virus, alphaviruses, and influenza virus (Fulton and Bailey). Mice are genetically better characterized than non-human primates and have a greater number of pharmacological endpoints or biomarkers. Neonatal mice, for instance, appear to be supremely sensitive to some human viral pathogens and have been successfully used as models. Although mice appear to be a model to the NVT for the screening of live attenuated influenza vaccines, and transgenic mice constitute the single model, as an alternative for residual neurovirulence testing in poliovirus vaccines., It is, however, cruel, equally cumbersome and the readouts have to be extrapolated to healthy human physiology.

Though various systems and methods exist for testing neurovirulence in vaccine safety, they are animal based, cruel, cumbersome, and involves PETA. Also, the test results are extrapolated to humans and have occupational hazards, and are undertaken only for regulatory submission.

FIG. 3, shows a process flow 300 of vaccine development. The process flow 300 involves steps of basic research 302, clinical trial 304, clinical study 306, and computational analysis 308. In one embodiment, the basic research 302 performs mechanistic analysis such as proof of concept. In one embodiment, in clinical trial 304 vaccine testing is performed. In one embodiment, the clinical trial 304 is performed to ensure safety and for efficacy biomarker identification. In one embodiment, the clinical study 306 performs natural identification such as biomarker identification. In one embodiment, the computational analysis 308 is used for analysis, validation, and modeling the result for the tested vaccines. in one embodiment, the basic research 302 and computation analysis 308 results in new hypothesis 310. In one embodiment, the clinical trial 304 and clinical study 306 can result in new products 312. In one embodiment, the process flow 300 for vaccine development is a vice-versa process. In one embodiment, the basic research 302 can be directed to a clinical study 306 and vice-versa. Similarly, the clinical trial 304 into the computational analysis 308 and vice-versa.

Part of the reason for the slow transition to alternative models is also a mindset, and the cost and effort implicit in the redesign and deployment of an industrial process, its validation, and the concomitant requirements for changes in regulatory policy. Incorporating the various experimental targeted neuroattenuation processes in industrial workflows that already turn out a form of the viral vaccine variant, for instance, will require substantial resources.

### Culture-Free and In-Vitro

Explorations with non-animal models have led to culture-free and in vitro alternatives. These have shown promise, albeit with limitations. Culture-free tests include genetic sequencing and polymerase chain reaction (PCR) based tests. The exact sequence alterations that cause the return of neurovirulence are not known, especially when these alterations vary continuously. Sequencing will detect known genetic markers of neurovirulence, but will miss those that are newly and stochastically arisen; it is therefore not definitive, and has utility only as a screening tool. Sequencing techniques have therefore been unable to stand in for culture-based techniques in evaluating neurovirulence; the MAPREC test for the Sabin live poliovirus vaccine is the best of few examples.

In vitro models are the most recent entrants to this area, and comprise immortalized cell lines, primary cell-lines, and stem cells of various types, including induced pluripotent stem cells (iPSCs). Their human genetic background is an advantage to become human surrogate invitro systems. Immortalized cell-lines diverge genetically from the original source and have other morphological and functional differences because of repeated passage in artificial culture, and therefore have limited predictive value, while primary progenitor cells, sourced from human biological discards, configured and co-axed to neuronal lineage as a platform is a reality to be utilized in developing cruelty-free in-vitro testing systems, modeled to be integrated with the workflow.

Various studies have demonstrated that hiPSCs and their derivatives have many of the qualities required of a model for neurotropic virus-host CNS interactions, so much so that they appear to be viable models for neuroattenuated live viral vaccines. hiPSCs-derived neuronal cells reproduce many of the features of CNS cells in vivo and have been found to have several of the key functional features of the nerve cells in the human body; the resemblance is deemed close enough for drug screening assays, and for evaluations of vaccine neurovirulence, to be relevant and viable; co-cultures of multiple stem cell types have occasionally been used to more closely mimic the human system. Their use in this capacity would still require extensive validation alongside the current standard-of-testing before they can receive regulatory approval.

Therefore, there is a need for a system and method that effectively tests neurovirulence and neurotoxicity in a vaccine composition for safety testing and prediction of risk in the vaccine batches produced for either clinical trials or for release into immunization program. Also, there is a need for system and method that includes a reliable testing mechanism for reacquisition of human neurovirulent specific readouts in the vaccine batch production. Further, there is a need for a system and method which involves a non-animal, rapid neurovirulence prediction test as a process-related quality check that is adopted by the global vaccine industry in the manufacturing stage.

### SUMMARY OF THE INVENTION

The present invention generally relates to the field of any structural or functional mimic of neurotropic virus or viral component developed as vaccines. More specifically, the present invention relates to a system and method for cruelty-free testing of safety risks such as neurovirulence, neurotoxicity, and sterility in vaccines production process.

According to the present invention, the system is a computer-implemented system executed in a network environment for testing neurovirulence in a vaccine aliquot. The system runs in the computer-implemented environment configured to provide a workstation solution that test predicts human neurovirulent signals. In one embodiment, the system utilizes a human biological discard sourced configured in vitro induced pluripotent stem cells-based platform (HuSu-TRANS-MSC) and a process automation enabled digital solution to build a digital human neurovirulence risk predicting solution. In one embodiment, the system is a cruelty-free digital human neurovirulence testing solution that monitors the Phenotype/Genotype/Proteotype inconsistency of healthy human stem cell-based platforms treated with vaccine aliquots. In one embodiment, the system performs a non-animal, rapid neurovirulence prediction test as a process-related quality check that is promoted to be adopted by the global vaccine industry. In one embodiment, the system involves a non-clinical safety assessment as a workflow process in evaluating neurovirulence during the research and development (R&D), clinical trials, and production for immunization programs.

In one embodiment, the system comprises a real-time platform or TRANS-MSC unit and the digital platform. The TRANS-MSC is a phenotypically responsive, genotypically reactive, functionally readable configured, characterized hiPSC based system, amenable to batch-wise large-scale production. In one embodiment, the TRANS-MSC is a human biological discard-sourced configured in vitro induced pluripotent stem cells-based microphysiological platform to build a digital neurovirulence testing podium. In one embodiment, the TRANS-MSC is configured to incubate the vaccine/biologic aliquots collected from the produced batches of vaccine.

In one embodiment, the digital platform or NeuroSAFE Software is embedded with one or more artificial intelligence (AI) and machine learning (ML) modules, augmented with a robotic process automation framework. The artificial intelligence modules are configured to predict neurovirulence, and by corollary, the degree of neuroattenuation of a vaccine capturing residual neurovirulence and neurotoxic signals along with any adventitious microbial contaminants in the test system. In one embodiment, the digital platform is trained with various human virus and bacteria-induced neurovirulent and neurotoxic cellular morphology patterns configured to develop a bandwidth for detecting the anomalies in real-time assaying. In one embodiment, the embedded AI and ML tools, augmented with Robotic Process Automation framework are trained with more than 1000 TRANS-MSC acquired phenotype micrographs and more than 250 barcoded neurotoxic genes involved in viral and bacterial infections.

In one embodiment, the network environment comprises one or more user devices. Each user device is associated with a user. In one embodiment, the user device is installed with a digital platform (i.e., NeuroSAFE software). In one embodiment, the digital platform may be an application software or mobile application or web-based application or software application. The system further comprises a network and a human neurovirulence evaluating system. In one embodiment, the user device is enabled to access the neurovirulence evaluating system via the network. In one embodiment, the user device enables the user to access one or more services provided by the system. In one embodiment, the user device is at least any one of a smartphone, a mobile phone, a tablet, a laptop, a desktop, and /or other suitable hand-held electronic communication devices. In one embodiment, the user device comprises a storage medium in communication with the network to access the neurovirulence evaluating system.

In one embodiment, the neurovirulence evaluating system comprises a computing device and one or more databases in communication with the computing device. In one embodiment, the computing device is a server. In one embodiment, the computing device could be a cloud server. In one embodiment, the database is in communication with the computing device via the network. In one embodiment, the database is accessible by the computing device. In one embodiment, the databases are configured to store a plurality of reference data.

In one embodiment, the computing device is configured to: extract phenotype images acquired on TRANS-MSC platform or data source treated with vaccine aliquot of the batch; map the extracted data with the functional annotation (AI/ML/NLP (Neural) with the reference data or training data sets; aggregate business rules for the extracted data, and visualize and analyze the extracted data by feeding into the software powered by machine learning algorithms that generate a score card and evaluates neurovirulence test and cellular infiltration. In one embodiment, the phenotype data points are acquired from images supported by respective genotype profiles run by the reference data.

In one embodiment, the batch needs to be discarded or recalled when the test material of the batch is found to be positive for the assay performed. In one embodiment, the score predicts human neurovirulent phenotype, cellular infiltrations, adverse events in clinics and microbiological contamination. In one embodiment, the system detects microbial contamination in the intermittent/finished batches. In one embodiment, the system is adopted to detect neurovirulence signals as clinical adverse events in pharmacovigilance.

In one embodiment, the method uses the system for testing neurovirulence prediction in the vaccine batch production. The system comprises a real-time platform or TRANS-MSC unit configured to incubate the vaccine/biologic aliquots collected from the produced batches of vaccine. The system further comprises a digital platform with embedded artificial intelligence (AI) and machine learning (ML) modules, augmented with a robotic process automation framework. The artificial intelligence modules are configured to predict neurovirulence, and by corollary, the degree of neuroattenuation of a vaccine along with any adventitious microbial contaminants in the test system.

The method comprises the following steps. At one step, the vaccine/biologic aliquots collected from the produced batches of vaccine are added into the TRANS-MSC unit seeded in a 6-well plate. At another step, the plate is incubated for a specified period in a CO2 incubator and the effects of the test material on the cells are recorded as phase-contrast microscopic images at the end of the incubation. At another step, a specified number of images are fed into the digital platform. At another step, the in vitro microphysiological platform is treated with the biologic, and digital platform is trained to discern between healthy and non-healthy morphologies. At another step, the biologically-affected in vitro system is graded into different categories. In one embodiment, the biologically-affected in vitro system are categorized into cells-in-shock, infiltrated, apoptotic, necrotic, and dead.

At another step, the damage is quantified and a score card is generated that is predictive of the biologic’s propensity for causing or triggering neurovirulence in vaccinated population, to quantify the neurovirulence potential for safety testing and prediction of risk. In one embodiment, the quantitative nature of the assay and the automation of the test process reduces the technical variability between measurements and allows comparison with neurovirulence measurements from other test formats. In one embodiment, the test is customized to a genetically distinct population, or to the user’s library of research-grade, clinical-grade raw materials, intermediates, APIs, final products that are at the risk of causing neurovirulence or neurotoxicity in the immunization programs. The quantification of the score of in-vitro human neurovirulence and cellular infiltration holds promise not only as a replacement for animal testing but as a measure of manufacturing consistency and freedom of adventitious contamination inducing debilitating neuropathy.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 shows the tendency or capacity of the vaccine to cause or trigger disease of the nervous system.

FIG. 2 shows a monkey neurovirulence test methodology (MNVT).

FIG. 3 shows a process flow for vaccine development.

FIG. 4 shows a computer-implemented system executed in a network environment for testing neurovirulence in a vaccine in an embodiment of the present invention.

FIG. 5 shows a schematic diagram of a smart vaccine testing platform in one embodiment of the present invention.

FIGS. 6-8 show morphological features followed by changes in gene expression status reveal in one embodiment of the present invention.

FIG. 9 shows a schematic diagram of transforming neurovirulence testing through digitalization in one embodiment of the present invention.

FIG. 10 shows a method for testing neurovirulence in the vaccine in one embodiment of the present invention.

FIG. 11 shows a screenshot of a user registration page of a digital platform in one embodiment of the present invention.

FIG. 12 shows a screenshot of a dashboard of the digital platform in one embodiment of the present invention.

FIG. 13 shows a screenshot of a report of the digital platform in one embodiment of the present invention.

FIG. 14 shows a screenshot of batch details in one embodiment of the present invention.

FIG. 15 shows a screenshot of a scorecard generation and evaluation of neurovirulence test in one embodiment of the present invention.

FIG. 16 shows a screenshot of automated image analysis in one embodiment of the present invention.

FIG. 17 shows a screenshot of the analyzed data of neurovirulence in one embodiment of the present invention.

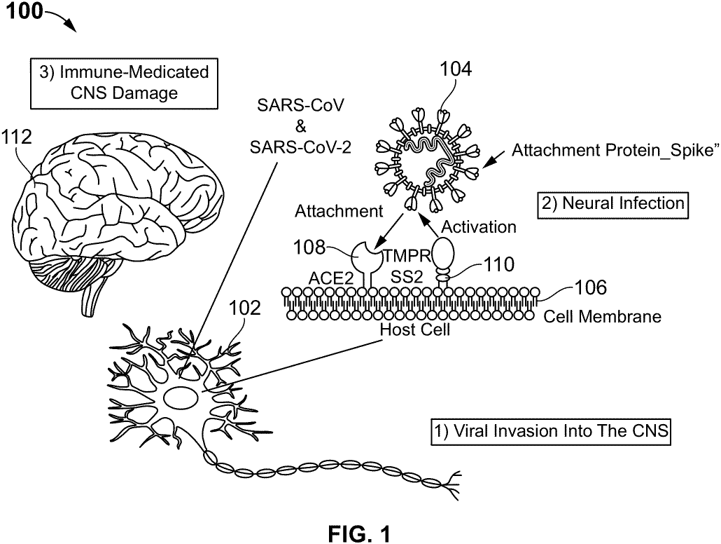

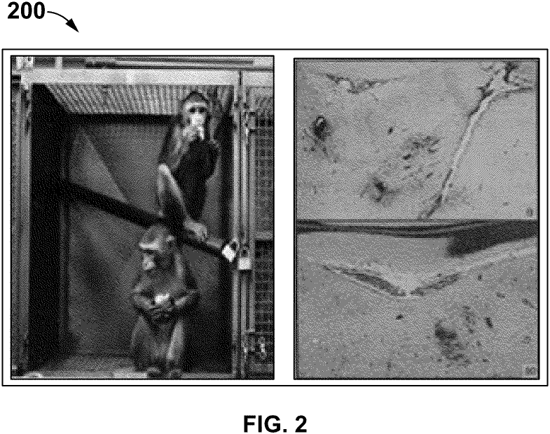

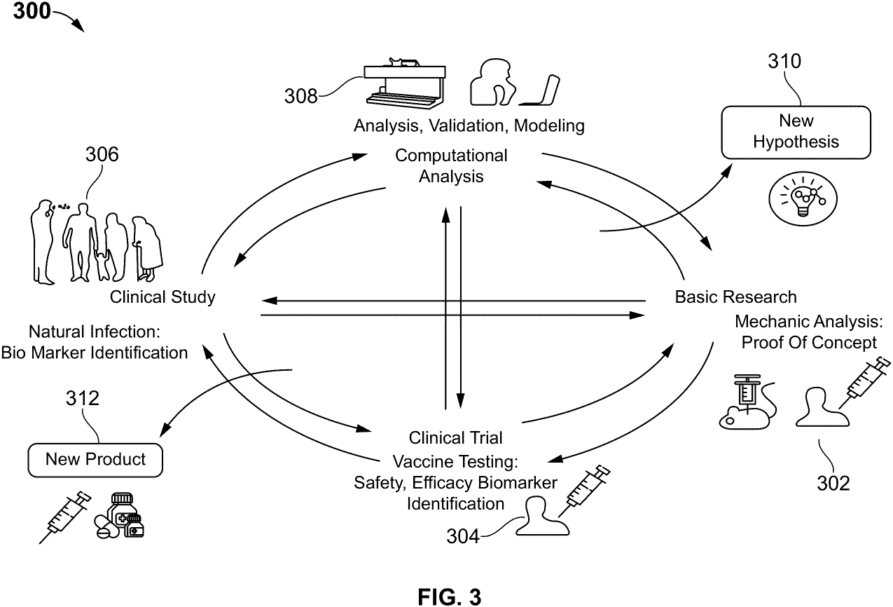

### DETAILED DESCRIPTION OF EMBODIMENTS

The present invention is best understood by reference to the detailed figures and description set forth herein.

It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

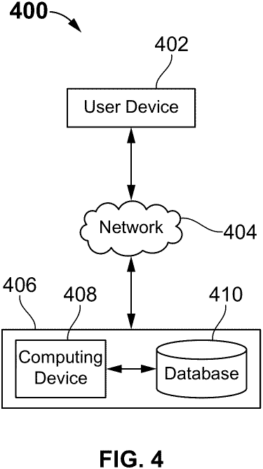

Referring to FIG. 4, a computer-implemented system executed in a network environment 400 for testing neurovirulence in a vaccine, according to one embodiment of the present invention. The system runs in the computer-implemented environment 400 configured to provide a workstation solution that test predicts neurovirulence. In one embodiment, the system utilizes a human biological discard sourced configured in vitro induced pluripotent stem cells-based platform (HuSu-TRANS-MSC) and a process automation enabled digital solution to build a digital neurovirulence test predicting solution. In one embodiment, the system is a cruelty-free digital neurovirulence solution that monitors the Phenotype/Genotype/Proteotype inconsistency of healthy human stem cell-based platforms treated with vaccine aliquots. In one embodiment, the system performs a non-animal, rapid neurovirulence prediction test as a process-related quality check that is encouraged to be adopted by the global vaccine industry. In one embodiment, the system involves a safety assessment as a workflow process in evaluating neurovirulence during the research and development (R&D), clinical trials, and production for immunization programs.

In one embodiment, the network environment 400 comprises one or more user devices 402. Each user device 402 is associated with a user. In one embodiment, the user device 402 is installed with a digital platform (i.e., NeuroSAFE software). In one embodiment, the digital platform may be an application software or mobile application or web-based application or software application. The system further comprises a network 404 and a neurovirulence evaluating system 406. In one embodiment, the user device 402 is enabled to access the neurovirulence evaluating system 406 via the network 404. In one embodiment, the user device 402 enables the user to access one or more services provided by the system. In one embodiment, the user device 402 is at least any one of a smartphone, a mobile phone, a tablet, a laptop, a desktop, and /or other suitable hand-held electronic communication devices. In one embodiment, the user device 402 comprises a storage medium in communication with the network 404 to access the neurovirulence evaluating system 406. In an embodiment, the network 404 could be Wi-Fi, WiMAX, wireless local area network (WLAN), satellite networks, cellular networks, private networks, and the like.

In one embodiment, the neurovirulence evaluating system 406 comprises a computing device 408 and one or more databases 410 in communication with the computing device 408. In one embodiment, the computing device 408 is a server. In one embodiment, the computing device 408 could be a cloud server. In one embodiment, the server could be operated as a single computer. In some embodiments, the computer could be a touchscreen and/or non-touchscreen and adopted to run on any type of OS, such as iOS™, Windows™, Android™, Unix™, Linux™, and/or others. In one embodiment, the plurality of computers is in communication with each other, via networks. Such communication is established via any one of an application software, a mobile application, a browser, an OS, and/or any combination thereof.

In one embodiment, the database 410 is in communication with the computing device 408 via the network 404. In one embodiment, the database 410 is accessible by the computing device 408. In another embodiment, the database 410 is integrated into the computing device 408 or separate from it. In some embodiments, the database 410 resides in a connected server or a cloud computing service. Regardless of location, the database 410 comprises a memory to store and organize certain data for use by the computing device 408.

In one embodiment, the computing device 408 comprises a processor and a computer-readable medium or memory unit coupled to the processor. The memory unit stores a set of instructions executable by the processor configured to test neurovirulence in the aliquots and to predict the risk involved. The memory unit could be RAM, ROM (including EPROM, EEPROM, PROM). In one embodiment, the user devices 402 are configured to access the services provided by the computing device 408 via the network 404. In one embodiment, the computing device 408 is configured to provide communication between the users in the digital platform.

In one embodiment, the computing device 408 is configured to, extract phenotype images acquired on TRANS-MSC platform or data source treated with vaccine aliquot of the batch; map the extracted data with the functional annotation (AI/ML/NLP (Neural) with the reference data or training data sets; aggregate business rules for the extracted data, and visualize and analyze the extracted data by feeding into the software powered by machine learning algorithms that generate a score card and evaluates human neurovirulence test and cellular infiltration. In one embodiment, the phenotype data points are acquired from images supported by respective genotype profiles run by the reference data.

In one embodiment, the batch needs to be discarded or recalled when the test material of the batch is found to be positive for the assay performed. In one embodiment, the score predicts human neurovirulent phenotype, cellular infiltrations, adverse events, and microbiological contamination. In one embodiment, the system detects microbial contamination in the intermittent/finished batches. In one embodiment, the system is adopted to detect neurovirulence signals in pharmacovigilance.

In one embodiment, the system comprises a real-time platform or TRANS-MSC unit and the digital platform. The TRANS-MSC is a phenotypically responsive, genotypically reactive, functionally readable configured, characterized hiPSC based system, amenable to batch-wise large-scale production. In one embodiment, the TRANS-MSC is a human biological discard-sourced configured in vitro induced pluripotent stem cells-based platform to build a digital neurovirulence solution. In one embodiment, the TRANS-MSC is configured to store the vaccine/biologic aliquots collected from the produced batches of vaccine.

In one embodiment, the digital platform or NeuroSAFE Software is embedded with one or more artificial intelligence (AI) and machine learning (ML) modules, augmented with a robotic process automation framework. The artificial intelligence modules are trained to predict neurovirulence, and by corollary, the degree of neuroattenuation of a vaccine along with any adventitious microbial contaminants in the test system. In one embodiment, the digital platform is trained with various human virus and bacteria-induced neurovirulent and related neurotoxic cellular morphology patterns configured to develop a bandwidth for detecting the anomalies in real-time assaying. In one embodiment, the embedded AI and ML tools, augmented with process automation framework are trained with more than 1000 TRANS-MSC acquired phenotype micrographs and more than 250 neurotoxic genes involved in viral and bacterial infections.

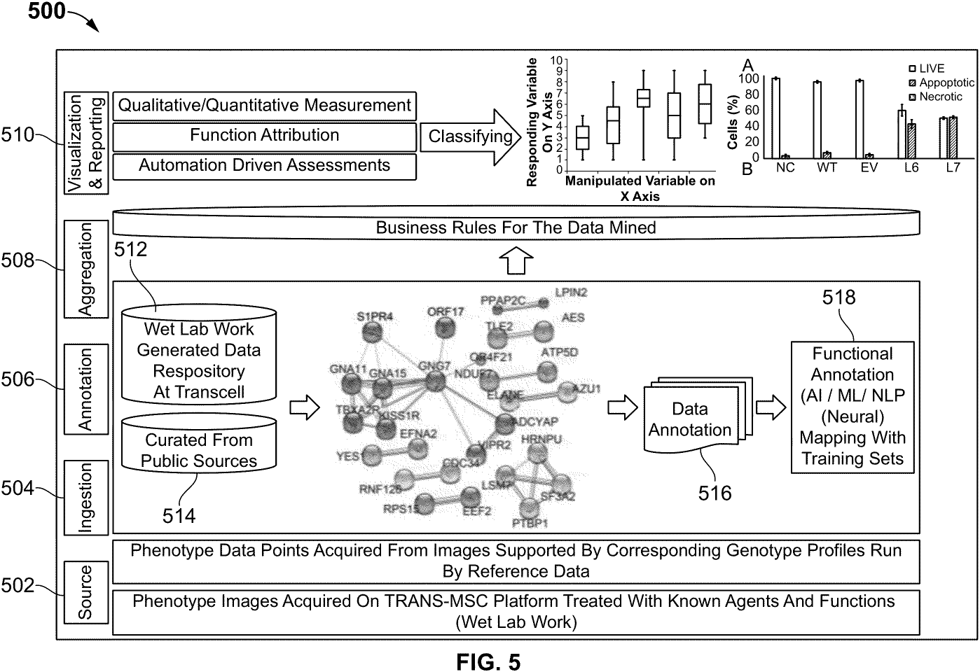

Referring to FIG. 5, a schematic diagram of a smart vaccine testing platform 500, according to one embodiment of the present invention. The smart vaccine testing platform 500 comprises one or more modules to perform neurovirulence testing. The smart vaccine testing platform 500 comprises a data source or source module 502, a data ingestion module or ingestion module 504, a data annotation module or annotation module 506, a data aggregation module or aggregation module 508, and a visualization and reporting module 510. In one embodiment, the data source 502 comprises a plurality of phenotype images and reference data. The phenotype images are acquired on TRANS-MSC platform that is treated with known agents and functions, for example, wet lab work. In one embodiment, the phenotype data points acquired from images supported by respective genotype profiles run by reference data.

In one embodiment, the acquired data is mapped with the reference data given in the training data sets using data ingestion module 504 and data annotation module 506. The data may be collected from wet lab generated data bank or web lab work generated data repository 512 at transcell. The data may be curated from public sources 514. These data are transferred for data annotation 516 via a middle source or physical entity for data mapping. In one embodiment, functional annotation mapping of data 518 is performed with reference data or training sets using AI/ML/NLP. In one embodiment, the data aggregation module 508 provides business rules for the extracted data. The extracted data is provided as an information matrix. In one embodiment, the visualization and reporting module 510 is configured to perform a qualitative/quantitative measurement, function attribution, and automation-driven assessments to classify quality profiling of data analysis in various formats such as reports, charts, and graphs.

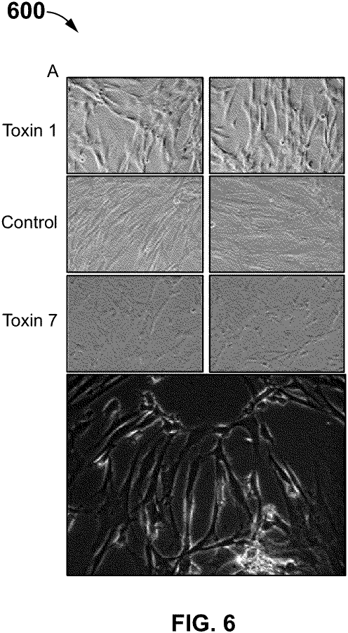

Referring to FIGS. 6-8, morphological features of the phenotypically and genotypically responsive hiPSC platform (TRANS-MSC) 600, according to one embodiment of the present invention. The digital platform reveals the morphological features followed by the changes in gene expression status. The digital platform draws on recent advances in stem-cell research and artificial intelligence to predict neurovirulence, and by corollary, the degree of neuroattenuation of a vaccine along with any adventitious microbial contaminants in the test system. It is possibly the first of a new class of in vitro assays that are substitutes for the conventional animal assay; a class of assays whose development has been impelled by ethical, scientific, and hard-headed economic concerns. While the cellular model may not reproduce the system’s level organization of the whole human body, they are nevertheless made sufficiently “close” to the “real thing” by complementing with an intelligent digital platform and becoming increasingly better by the day on Kaizen key embedded in the system. Extensively used in the study of virus-host interactions, TRANS-MSC platform/hiPSC systems have been demonstrated to reproduce various aspects of the neuronal cells in a whole organism. In retrospect, it is only logical and appropriate that they be used for assaying human neurovirulence.

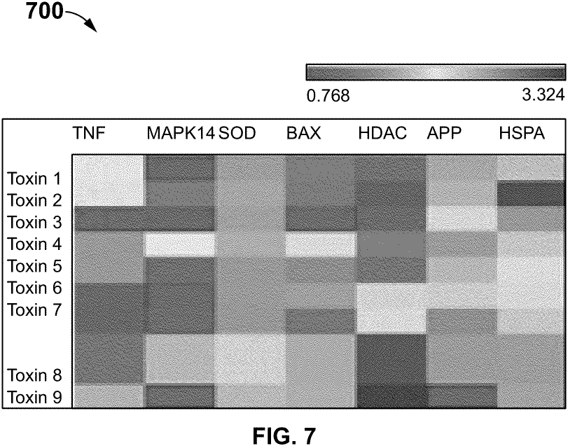

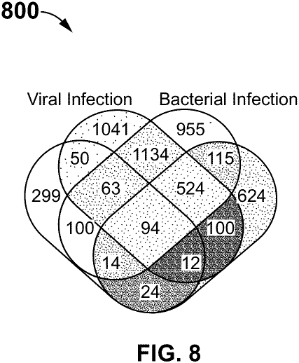

The TRANS-MSC platform is sensitive to test predict human neurovirulent phenotypes. In one embodiment, the TRANS-MSC is a phenotypically responsive, genotypically reactive, functionally readable configured, characterized hiPSC based system, amenable to batch-wise large-scale production. The TRANS-MSC is tested with various toxins at different time intervals. In one embodiment, the TRANS-MSC is treated with 5 different concentrations and 3 different time intervals. The TRANS-MSC is tested with various pictographs and neurotoxic gene sets that are stored in the web lab work data bank 512. The web lab work data bank 512 comprises more than 1000 pictographs with phase-contrast micrographs of virus, vaccine, bacteria, toxin treated TRANS-MSC, and more than 250 neurotoxic gene sets that have established roles in chemical, bacterial, viral-induced neurotoxicity pathways. For example, the neurovirulent phenotype of the human corona virus-infected TRANS-MSC is shown in FIG. 6. The changes in gene expression status 700 are shown in different color variations in FIG. 7. Further, the neurotoxicity due to the viral infection and bacterial infection 800 are shown in FIG. 8.

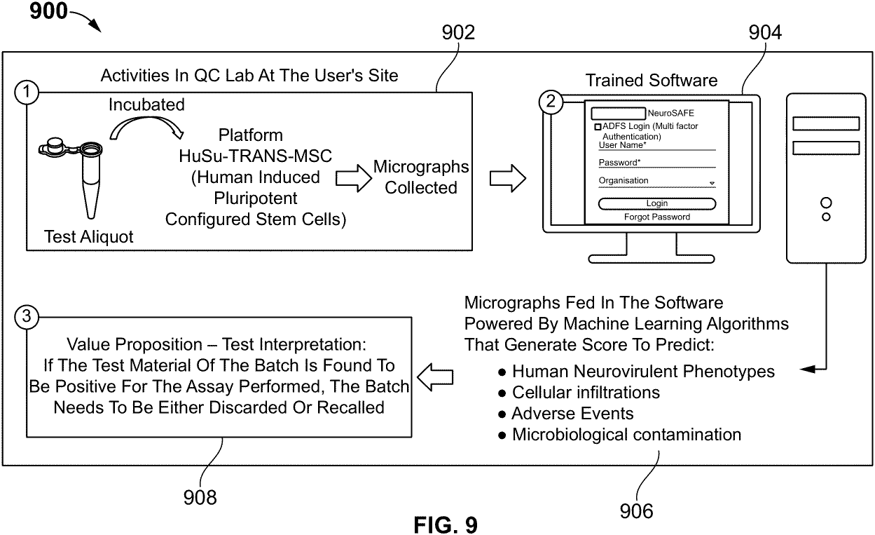

Referring to FIG. 9, a block diagram 900 for transforming neurovirulence testing through digitalization, according to one embodiment of the present invention. The block diagram 900 comprises a data foundation module 902 that performs data extraction and simulation to optimize and interconnect one or more data. In one embodiment, the aliquots collected from the batches are added to the TRANS-MSC unit/TRANS-MSC platform that is seeded in a 6-well plate. In one embodiment, the plate is incubated for a specified period in an incubator and the effects of the test material on the cells are recorded as phenotype data or phase-contrast microscopic images at 20X at the end of the incubation. The collected phenotype data is fed into a digital analytics-data-driven module 904, which orchestrates the collected data. In one embodiment, the module 904 is installed with the application software or digital platform. The digital platform is configured to perform image analysis, molecular analysis, and provides data analytics solutions. The data is then fed into the application software powered by artificial intelligence and machine learning algorithms 906 configured to generate a scorecard. The scorecard predicts human neurovirulence phenotype, cellular infiltrations, adverse events, and microbiological contamination.

In one embodiment, the block diagram 900 further comprises a report transformation module 908 that provides data-centric neurovirulence reports. In one embodiment, the transformation module 908 provides more personalized and monetized report. In one embodiment, the transformation module 908 performs test interpretation. If the test material of the batch is found to be positive for the assay performed, the batch needs to be discarded or recalled.

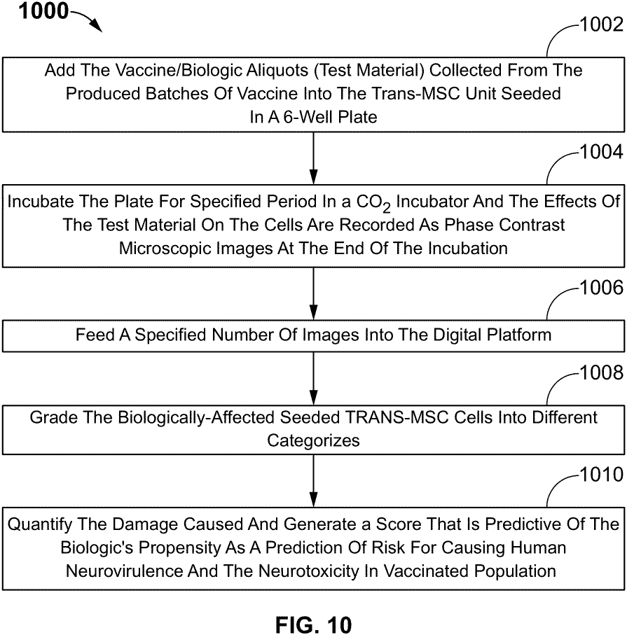

Referring to FIG. 10, a method 1000 for testing neurovirulence in a vaccine, according to one embodiment of the present invention. The method 1000 uses a computer-implemented system. The system comprises a real-time platform or TRANS-MSC unit configured to store the vaccine/biologic aliquots collected from the produced batches of vaccine. The system further comprises a digital platform with embedded artificial intelligence (AI) and machine learning (ML) modules, augmented with a robotic process automation framework. The artificial intelligence modules are configured to predict neurovirulence, and by corollary, the degree of neuroattenuation of a vaccine along with any adventitious microbial contaminants in the test system.

The method 1000 comprises the following steps. At step 1002, the vaccine/biologic aliquots (test material) collected from the produced batches of vaccine are added into the TRANS-MSC unit seeded in a 6-well plate. At step 1004, the plate is incubated for a specified period in a CO2 incubator and the effects of the test material on the cells are recorded as phase-contrast microscopic images at the end of the incubation. At step 1006, a specified number of images are fed into the digital platform. The test cells are treated with the biologic, and healthy cells to train the system to discern between test cells with different morphology. At step 1008, the biologically-affected seeded TRANS-MSC cells are graded into different categorizes. In one embodiment, the biologically-affected seeded TRANS-MSC cells are categorized into cells-in-shock, infiltrated, apoptotic, necrotic, and dead.

At step 1010, the damage is quantified and a score card is generated that is predictive of the biologic’s propensity for causing neurovirulence to quantify the neurovirulence potential for safety testing and prediction of risk. In one embodiment, the quantitative nature of the assay and the automation of the test process reduces the technical variability between measurements and allows comparison with neurovirulence measurements from other test formats. In one embodiment, the test is customized to a genetically distinct population, or to the user’s library of research-grade, clinical-grade raw materials, intermediates, APIs, final products that are at the risk of causing neurovirulence or neurotoxicity in the immunization programs. The quantification of the score of in-vitro neurovirulence and cellular infiltration holds promise not only as a replacement for animal testing but as a measure of manufacturing consistency and freedom of adventitious contamination inducing debilitating neuropathy.

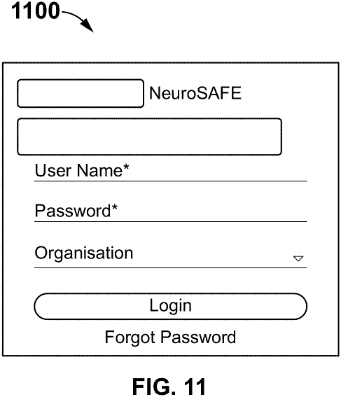

Referring to FIG. 11, a screenshot 1100 of a user registration page of the digital platform, according to one embodiment of the present invention. In one embodiment, the digital platform or NeuroSAFE may be a dedicated application software or mobile application or web-based application or desktop application. The application software allows the user to register into it using one or more user credentials such as first name, last name, user name, email id, and password. Upon successful registration, the application software collects one or more login credentials such as user name, password, and organization detail to enter into the application software. The application software also allows the user to change their password using “Forget Password” option.

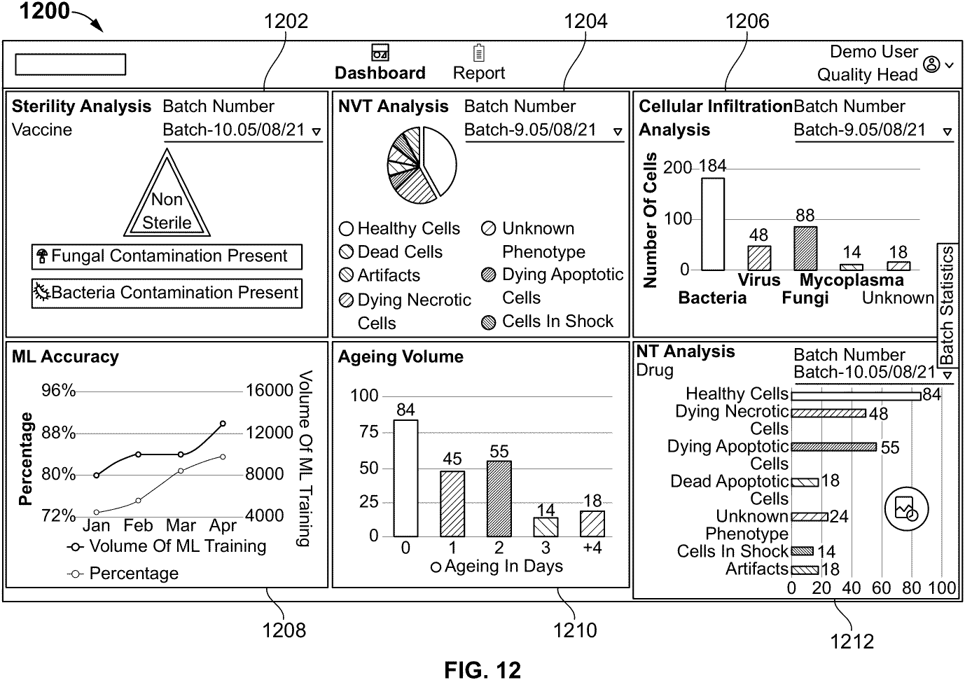

Referring to FIG. 12, a screenshot 1200 of a user dashboard of the digital platform, according to on embodiment of the present invention. The dashboard may include one or more graphical representation of various analyses. The various analyses include, but are not limited to, sterility analysis 1202, NVT analysis 1204, cellular infiltration analysis 1206, ML accuracy analysis 1208, aging volume analysis 1210, and NT analysis 1212. The sterility analysis 1202 provides details about fungal contamination and bacteria contamination present in the vaccine. The NVT analysis 1204 grades test cells into different categories such as healthy cells, dead cells, artifacts, dying necrotic cells, unknown phenotype, dying apoptotic cells, and cells in shock. The cellular infiltration analysis 1206 provides the details of bacteria, viruses, fungi, mycoplasma, and unknown microbes in the given number of test cells. The ML accuracy analysis 1208 provides a volume of ML training and percentage. The aging volume analysis 1210 provides the details of produced batches and aging of vaccine in days. The NT analysis 1212 provides the percentage details of healthy cells, dying necrotic cells, dying apoptotic cells, dead apoptotic cells, unknown phenotype, cells-in-shock, and artifacts present in the test material.

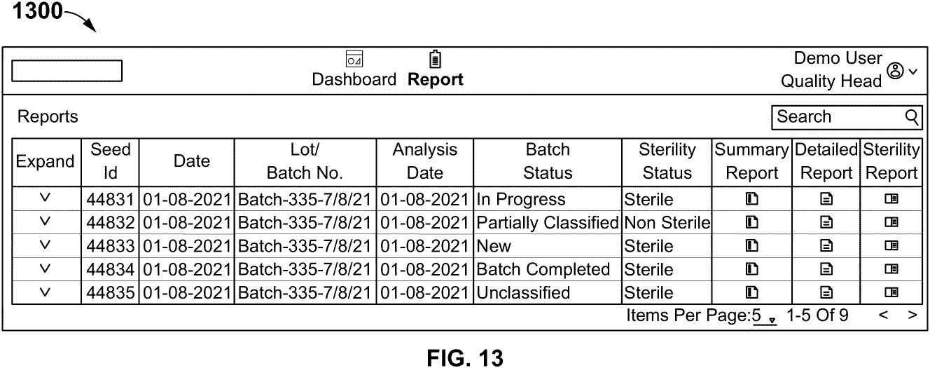

Referring to FIG. 13, a screenshot 1300 of a report option of the digital platform, according to one embodiment of the present invention. The report comprises a list of reports for the vaccine. Each report includes a seed id, data, lot/batch number, analysis date, batch status (for example, in progress, partially classified, new, batch completed, or unclassified), sterility status, summary report, detailed report, and sterility report. The user may expand the report on the same page without leaving to another page using an “Expand” option.

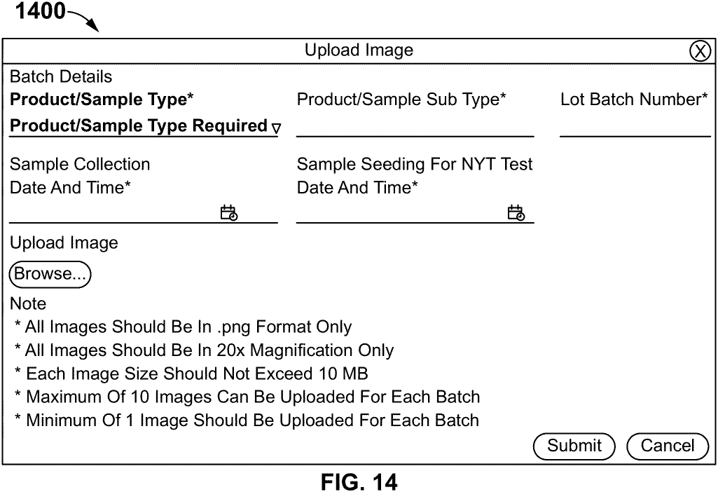

Referring to FIG. 14, a screenshot 1400 of a portal for uploading image and image details, according to one embodiment of the present invention. In one embodiment, the portal requests batch details of the image such as Product/sample type, product/sample sub-type, lot/batch number, sample collection date and time, and sample seeding for NVT test date and time. In one embodiment, the product/sample type is selected from the dropdown list provided. In one embodiment, the date and time are selected from the calendar integrated with the portal. The portal further requests images for uploading. In one embodiment, the images could be uploaded by browsing. The portal also displays a note for uploading the image in the portal. In on embodiment, the note includes details such as “All images should be in .png format only”, “All images should be in 20x magnification only”, “Each image size should not exceed 10MB”, “Maximum of 10 images can be uploaded for each batch”, and “Minimum of 1 image can be uploaded for each batch”. The portal further includes submit and cancel options. The user can submit or cancel the uploaded details as per their preference.

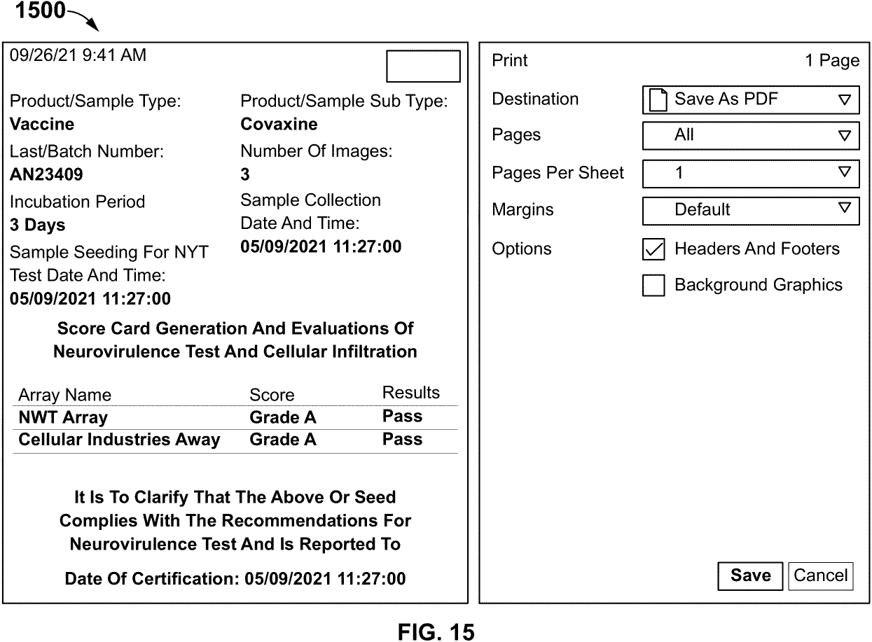

Referring to FIG. 15, a screenshot 1500 of a document with sample details, according to one embodiment of the present invention. In one embodiment, the document includes details of the images uploaded. In one embodiment, the details include product/sample type, product/sample sub-type, Lot/batch number, number of images uploaded, incubation period, sample collection date and time, and sample seeding for NVT test date and time. In one embodiment, the document further shows details of score-card generation and evaluations of neurovirulence test and cellular infiltration. In one embodiment, the score-card generated includes details such as array name, scores in grade, and results i.e., pass or fail. In one embodiment, the document states that “It is to clarify that above or seed complies with recommendations for neurovirulence test and is reported to date of certification”. In one embodiment, the document further includes certification date and time. In one embodiment, the certification can be printed or saved as PDF in the required folder.

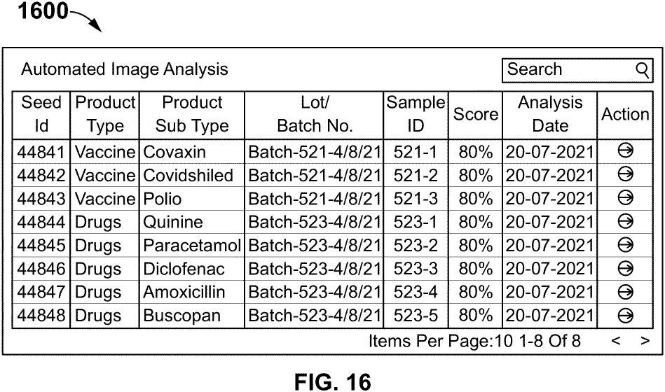

Referring to FIG. 16, a screenshot 1600 of automatic image analysis for neurovirulence in the vaccine, according to one embodiment of the present invention. In one embodiment, the analysis shows a table listing with seed Id, product type either vaccine or drug, product sub-type, Lot/batch number, sample ID, scores in percentage, analysis date and actions to be taken. In one embodiment, the product sub-type includes Covaxin, Covidshield, Polio, Quinine, Paracetomal, Diclofence, Amoxiline, Buscopan, and other types of products. In one embodiment, the score is generated based on quantifying the damage caused in the cell. In one embodiment, the analysis is also provided with a search bar at the top for search specific items in the list. In one embodiment, it displays the number of items per page at the bottom along with forward and backward arrows for moving.

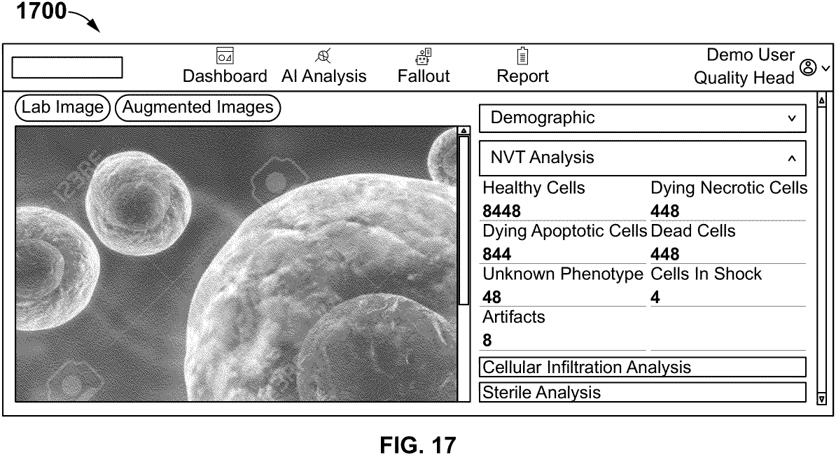

Referring to FIG. 17, a screenshot 1700 of the analyzed data of neurovirulence, according to one embodiment of the present invention. In one embodiment, after login into the workstation using user login credentials, the workstation display user name and their position along with their profile image i.e., demo user, quality head. In one embodiment, the workstation also displays an analyzed image. In one embodiment, the image could be a lab image or an augmented image. In one embodiment, the workstation further displays details of demographic data, NVT analysis, cellular infiltration analysis, and sterile analysis data. In one embodiment, the NVT analysis includes details of the number of healthy cells, dying necrotic cells, dying apoptotic cells, dead cells, unknown phenotype, cells in stock, and artifacts. In one embodiment, the workstation also has other options for processing the captured image such as dashboard, AI analysis, Fallout, and report.

In one embodiment, the digital platform is designed for rapid and easy deployment and integration into the manufacturer’s workflow or standard operating protocols. It brings benefits that make its adoption worthwhile. The system requires reagents that are easily available and inexpensive and does not require additional training. These aren’t benefits to scoff at as changes in workflows cost companies time, money, and effort, in addition to creating an interruption in productivity. The system is sufficiently simple to find acceptance in an industry cautious to change. It does not require animal models or biopsies and is in line with cruelty-free practices. It expedites testing, from 28 days to a few hours, and can be scaled as desired.

In one embodiment, the system is integrated into the user’s workflow without the need for test material leaving the production premises to any animal house facilities, the quantitative nature of the assay, the automation of the process reduces the technical variability between measurements and allows comparison with neurovirulence measurements from other test formats. The test can be customized to a genetically distinct population, or to the user’s library of research-grade, clinical-grade raw materials, intermediates, APIs, final products that are at the risk of causing Neurovirulence or Neurotoxicity in the immunization programs. Further, the system’s testing strategy will evolve continuously over time. As basic research on virus-host interactions advances and knowledge of these interactions improves, newer cellular and molecular end-points for neurovirulence will be revealed, and can be incorporated into the system; multiple end-points enable finer analyses. The system’s algorithm can be made more intelligent and reliable through continuous input data circulation from its adoption viz by triangulation and kaizen key.

In one embodiment, the system may be used in other industries such as biologics (proteins, antibodies, CAR-T cells), generics/new drugs (small molecules), APIs, and anti-venom. It is a fundamental requirement that all venoms and anti-venoms batches produced are tested for their safety and efficacy - initially by LD50 and then by ED50 in mice. A number of in vitro approaches have employed cell-based assays to determine the effects of test compounds on cell viability. Despite the compositional complexities of snake venoms, an assay testing system could be an interesting alternative in both LD50 and ED50 like potency calculations paying special attention to increasing public concern for animal welfare as alternative non-animal-based toxicity assays.

According to the present invention, the system performs the test to predict safety risks, which comprises the following steps: At one step, a memorandum of understanding between the digital platform and the client is entered with an expression of interest in adopting the digital platform through NeuroSAFE Acclimatization Period (NAP) in their workflow. At another step, requirements are gathered on the number of clients’ products to train the digital platform with product specific Neurovirulence and Neurotoxic patterns. At another step, the selected products are treated at various concentrations on the real-time platform TRANS-MSC to generate phenotypic micrographs (n=500 each) at the client’s site. At another step, the software is implemented and onboard in the client’s IT system. At another step, the user acceptance is tested. At another step, the designated personnel on handling TRANS-MSC and the digital platform is trained. At another step, the contract period begins in real-time with the system adoption in the client’s quality control/assurance department to test predict safety risks like neurovirulence, neurotoxicity, sterility in the batches produced as a routine process.

Advantageously, the system of the present invention performs a non-animal, rapid neurovirulence prediction test as a process-related quality check that is adopted by the global vaccine industry. The system is compatible with all kinds of Covid-19 vaccines, for example, structural and functional mimics of virus and the subunits. The system is suitable for other vaccines that are mandated to be tested for neurovirulence as per the global regulatory guidelines. The system is suitable for detecting microbial contamination in the intermittent/finished batches. Also, the system can be adopted to detect neurovirulence signals in pharmacovigilance. Further, the system is used in other industries such as biologics (proteins, antibodies, CAR-T cells), generics/new drugs (small molecules), APIs, and anti-venom. The WHO mandates the development and adoption of in vitro alternatives to the in vivo assays to reduce the number of animals used in the vaccine and anti-venom industry.

Preferred embodiments of this invention are described herein, including the best mode known to the inventors for carrying out the invention. It should be understood that the illustrated embodiments are exemplary only and should not be taken as limiting the scope of the invention.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings in the foregoing descriptions. Although specific terms may be employed herein, they are used only in generic and descriptive sense and not for purposes of limitation. Accordingly, the present invention is not limited to the specific embodiments illustrated herein.

We claim:

1. A computer-implemented system for testing human neurovirulence in a vaccine comprising:
a real-time platform or TRANS-MSC (Human Mesenchymal Stem Cells) unit configured to incubate the vaccine/biologic aliquots collected from the produced batches of vaccine, and
a digital platform with embedded artificial intelligence (AI) and machine learning (ML) modules, augmented with robotic process automation framework, wherein the artificial intelligence modules are configured to predict neurovirulence, and by corollary, the degree of neuroattenuation of a vaccine along with any adventitious microbial contaminants in the test system,
wherein the digital platform is trained with various human virus and bacteria-induced neurovirulent and neurotoxic cellular morphology patterns configured to develop a bandwidth for detecting the anomalies in real-time assaying.

2. A computer-implemented system for test predicting induced human neurotoxicity in a drug comprising:
a real-time platform or TRANS-MSC unit configured to incubate the drug/API aliquots collected from the produced batches and
a digital platform with embedded artificial intelligence (AI) and machine learning (ML) modules, augmented with robotic process automation framework, wherein the artificial intelligence modules are configured to predict neurotoxicity of a drug/API along with any adventitious microbial contaminants in the test system,
wherein the digital platform is trained with various human virus, mycoplasma like fungi
and bacteria-induced neurovirulent and neurotoxic cellular morphology patterns configured to develop a bandwidth for detecting the anomalies in real-time assaying.

3. A computer-implemented system for test predicting induced human neurotoxicity in a cosmetic product comprising:
a real-time platform or TRANS-MSC unit configured to incubate the cosmetic chemical/ingredient aliquots collected from the produced batches and
a digital platform with embedded artificial intelligence (AI) and machine learning (ML) modules, augmented with robotic process automation framework, wherein the artificial intelligence modules are configured to predict neurotoxicity of a drug/API along with any adventitious microbial contaminants in the test system,
wherein the digital platform is trained with various human virus, mycoplasma like fungi
and bacteria-induced neurovirulent and neurotoxic cellular morphology patterns configured to develop a bandwidth for detecting the anomalies in real-time assaying.

4. A computer-implemented system for test predicting induced human neurotoxicity in a natural product comprising:
a real-time platform or TRANS-MSC unit configured to incubate the natural product/it’s API aliquots collected from the produced batches and
a digital platform with embedded artificial intelligence (AI) and machine learning (ML) modules, augmented with robotic process automation framework, wherein the artificial intelligence modules are configured to predict neurotoxicity of a drug/API along with any adventitious microbial contaminants in the test system,
wherein the digital platform is trained with various human virus, mycoplasma like fungi
and bacteria-induced neurovirulent and neurotoxic cellular morphology patterns configured to develop a bandwidth for detecting the anomalies in real-time assaying.

5. A computer-implemented system for test predicting induced human neurotoxicity in a cell based druggable candidate comprising:
a real-time platform or TRANS-MSC unit configured to incubate the cell based drug culture supernatant collected from the produced batches and
a digital platform with embedded artificial intelligence (AI) and machine learning (ML) modules, augmented with robotic process automation framework, wherein the artificial intelligence modules are configured to predict neurotoxicity of a drug/API along with any adventitious microbial contaminants in the test system,
wherein the digital platform is trained with various human virus, mycoplasma like fungi
and bacteria-induced neurovirulent and neurotoxic cellular morphology patterns configured to develop a bandwidth for detecting the anomalies in real-time assaying.

6. The system of claim 1-5, wherein the TRANS-MSC is a phenotypically responsive, genotypically reactive, functionally readable configured, characterized hiPSC based system, amenable to batch-wise large-scale production.

7. The system of claims 1-5, wherein the artificial intelligence and machine learning modules are trained with a plurality of TRANS-MSC acquired phenotype micrographs and a plurality of neurotoxic genes involved in viral, bacterial, fungal infections.

8. The system of claims 1-5, wherein the aliquots collected from the batches are added to the TRANS-MSC unit seeded in a 6-well plate.

9. The system of claims 1-5, wherein the aliquots collected from the batches are added to the TRANS-MSC unit seeded in a 96-well plate.

10. The system of claims 8-9, wherein the plate is incubated for a specified period in an incubator and the effects of the test material on the cells are recorded as phase-contrast microscopic images at the end of the incubation period.

11. The system of claims 1-5, wherein the digital platform is loaded with a number of specified images to train the system to discern between cells with different morphology as a result of treating them with the test material.

12. A system for testing neurovirulence or neurotoxicity or neurovirulence and neurotoxicity of claims 1-5, comprising:
a computing device having at least one processor and a memory in communication with the processor, wherein the memory stores a set of instructions executable by the processor;
one or more databases in communication with the computing device via a network configured to store a plurality of reference data, and
a user device associated with a user in communication with the computing device via the network configured to fed or upload an image data for analysis, wherein the computing device is configured to,
extract phenotype images acquired on TRANS-MSC platform or data source treated with vaccine aliquot of the batch, wherein the phenotype data points acquired from images supported by respective genotype profiles run by the reference data;
map the extracted data with the functional annotation (AI/ML/NLP (Neural) with the reference data or training data sets;
aggregate business rules for the extracted data, and
visualize and analyze the extracted data by feeding into the software powered by machine learning algorithms that generate a scorecard and evaluate neurovirulence test and cellular infiltration.

13. The system of claim 12, wherein the batch needs to be discarded or recalled when the test material of the batch is found to be positive for the assay performed.

14. The system of claim 12, wherein the score predicts human neurovirulent phenotype, human neurotoxic cellular phenotype, cellular infiltrations, adverse events, and microbiological contamination.

15. The system of claim 12, wherein the digital platform is application software or mobile application or web-based application or desktop application.

16. The system of claim 12, detects microbial contamination in the intermittent/finished batches and generates sterility report.

17. The system of claim 12, is adopted to detect neurovirulence signals in pharmacovigilance.

18. A method for test predicting human neurovirulence, human neurotoxic risks in a vaccine, drug, cosmetic, anti-venom products using a computer-implemented system having a real-time in vitro cell based platform or TRANS-MSC unit configured to incubate the test material’s aliquots collected from the produced batches, and a digital platform with embedded artificial intelligence (AI) and machine learning (ML) modules, augmented with robotic process automation framework, wherein the artificial intelligence modules are configured to predict neurovirulence, neurotoxicity patterns along with any adventitious microbial contaminants in the test system, wherein the method comprising the steps of:
adding the test material collected from the produced batches into the TRANS-MSC unit seeded in a 6-well plate or 96-well plate;
incubating the plate for a specified period in a CO2 incubator and the effects of the test material on the cells are recorded as phase-contrast microscopic images at the end of the incubation;
feeding a specified number of images into the digital platform;
grading the cells into different categories, and
quantifying the damage caused and generating a score that is predictive of the test material’s′ s propensity for causing human neurovirulence, neurotoxicity to humansto quantify the deleterious potential for safety testing and prediction of risk.

19. The method of claim 18, wherein the affected cells are categorized into cells-in-shock, infiltrated, apoptotic, necrotic, and dead.

20. The method of claim 18, wherein the quantitative nature of the assay and the automation of the test process reduces the technical variability between measurements and allows comparison with neurovirulence measurements from other test formats without the need of either positive control or negative control in the method.

21. The method of claim 20, wherein the test is customized to a genetically distinct population, or to the user’s library of research-grade, clinical-grade raw materials, intermediates, APIs, final products that are at the risk of causing neurovirulence or neurotoxicity in the clinics.
